(19) United States
(12) Patent Application Publication
(10) Pub. No.: US 2026/0126436 A1
(43) Pub. Date: May 7, 2026

(54) AMPLIFYING BIOMOLECULAR SENSING THROUGH ACOUSTICALLY ENHANCED REACTIONS

(71) Applicants: THE TRUSTEES OF COLUMBIA UNIVERSITY IN THE CITY OF NEW YORK, New York, NY (US); BROOKHAVEN SCIENCE ASSOCIATES, LLC, Upton, NY (US)

(72) Inventors: Oleg Gang, Setauket, NY (US); Feiyue Teng, Franklin, MA (US); Zohar Albert Arnon, New York, NY (US)

(73) Assignees: THE TRUSTEES OF COLUMBIA UNIVERSITY IN THE CITY OF NEW YORK, New York, NY (US); BROOKHAVEN SCIENCE ASSOCIATES, LLC, Upton, NY (US)

(21) Appl. No.: 19/379,879

(22) Filed: Nov. 5, 2025

Related U.S. Application Data
(60) Provisional application No. 63/716,794, filed on Nov. 6, 2024.

(51) Int. Cl. G01N 33/543; B01L 3/00; G01N 33/53; G01N 33/569

(52) U.S. Cl. CPC G01N 33/54333; B01L 3/502761; G01N 33/5308; G01N 33/54373; G01N 33/56983; B01L 2200/0668; B01L 2300/0663; B01L 2400/0439; G01N 2333/165

(57) ABSTRACT

The disclosed subject matter provides methods and apparatus for detecting a target analyte in a sample. The method includes providing nanoparticles functionalized with one or more recognition molecules configured to bind specifically to the target analyte; exposing the sample containing the target analyte and the functionalized nanoparticles to an acoustic field comprising a surface standing acoustic wave (SSAW); inducing aggregation of the nanoparticles mediated by the target analyte under acoustic stimulation of SSAW; and detecting the aggregation of the nanoparticles as an indication of the presence of the target analyte.

### CROSS-REFERENCE TO RELATED APPLICATION

This Application claims priority to U.S. Provisional Patent Application Ser. No. 63/716,794, filed Nov. 6, 2024, which is hereby incorporated by reference in its entirety.

### GRANT INFORMATION

This invention was made with government support under DE-SC0012704 awarded by the U.S. Department of Energy and W911NF-22-2-0111 awarded by the Army Research Office. The government has certain rights in the invention.

### BACKGROUND

The disclosed subject matter relates generally to biosensing and diagnostic technologies, and more particularly to techniques for amplifying biomolecular sensing through acoustically enhanced reactions.

Point-of-care diagnostic techniques are increasingly important tools for rapid detection of molecular biomarkers in healthcare, food safety, environmental monitoring, and related fields. A limitation of certain methods is their sensitivity, which can be constrained by the inherent kinetics of molecular recognition processes.

For example, molecular recognition between a target analyte and a receptor (e.g., antibody, aptamer, or enzyme) often proceeds slowly and can require relatively high analyte concentrations to achieve reliable detection due to a kinetic barrier, thereby limiting applicability in low-abundance biomarker scenarios, such as early-stage disease diagnostics.

Therefore, there is a need for developing techniques to overcome the limitations in biomolecular recognition in biosensors.

### SUMMARY

The disclosed subject matter provides techniques for a broad and reliable detection of target biomolecules, enabling the detection of target biomolecules of small cluster sizes and low concentrations. In particular, the disclosed subject matter employs acoustic wave energy to accelerate molecular recognition kinetics in nanoparticle-based biosensors, thereby significantly increasing detection sensitivity across a wide range of target analytes and sensing platforms.

An example method comprises providing nanoparticles functionalized with one or more recognition molecules configured to bind specifically to the target analyte, exposing the sample containing the target analyte and the functionalized nanoparticles to an acoustic field comprising a surface standing acoustic wave (SSAW), inducing aggregation of the nanoparticles mediated by the target analyte under acoustic stimulation of the SSAW, and detecting the aggregation of the nanoparticles as an indication of the presence of the target analyte.

In certain embodiments, the acoustic field is generated by applying an electrical signal to a piezoelectric substrate, such as lithium niobate (LiNbO₃), through a plurality of interdigital transducers (IDTs). The acoustic field can be configured to generate excitation at frequencies between about 1 MHz and about 50 MHz. In certain embodiments, acoustic stimulation is applied in pulses having a duration of about 10 ms to about 100 ms with a period of about 0.1 s to about 5 s. The pulse duration and acoustic period can be tuned to adjust aggregation efficiency.

In certain embodiments, the recognition molecules conjugated to the nanoparticles can include at least one of antibodies, antibody fragments, aptamers, nucleic acids, or enzymes. The target analyte can include at least one of DNA linker, protein, nucleic acid, pathogen, toxin, metabolite, or biomarker associated with a disease state. In certain embodiments, nanoparticles are functionalized with two or more recognition molecules that bind to non-overlapping epitopes of the analyte.

In certain embodiments, the detecting comprises at least one of optical detection, dynamic light scattering, dark-field microscopy, spectrophotometry, or electron microscopy. The SSAW is generated by the IDTs patterned on a piezoelectric substrate. The method further can comprise tuning pulse duration and acoustic period to adjust aggregation efficiency. The detecting comprises monitoring a plasmonic shift in optical spectra associated with aggregation of the nanoparticles.

In another aspect, the disclosed subject matter provides an apparatus for detecting a target analyte in a sample. An example apparatus can include a reaction chamber comprising a capillary configured to receive a sample containing a target analyte and nanoparticles functionalized with recognition molecules; a piezoelectric substrate operatively coupled to the reaction chamber; an electrical signal generator configured to apply an electrical signal to the piezoelectric substrate to generate a SSAW within the capillary; and a detector configured to measure aggregation of the nanoparticles mediated by the target analyte.

In certain embodiments, the detector can include an optical detector, spectrophotometer, dark-field microscope, or dynamic light scattering instrument. The detector can be configured to detect changes in hydrodynamic radius of nanoparticle clusters or to output a visual indication of aggregation. The nanoparticles can include gold nanoparticles conjugated to two or more antibodies recognizing specific epitopes of the target analyte.

In certain embodiments, the apparatus includes a lithium niobate (LiNbO₃) substrate, and the capillary comprises a glass capillary mounted directly on the substrate. The electrical signal generator can apply sinusoidal electrical signals to the interdigital transducers (IDTs) to generate counter-propagating acoustic waves, with the electrical signals further configured to vary frequency, amplitude, or duty cycle to adaptively tune the SSAW for different analytes or fluidic conditions.

### BRIEF DESCRIPTION OF THE DRAWINGS

Embodiments of the disclosed subject matter are described in detail by reference to the following drawings:

FIG. 1A depicts an illustrative acoustic stimulation apparatus according to an embodiment of the disclosed subject matter. FIG. 1B illustrates motions and aggregation propelled by acoustic waves.

FIG. 2 depicts sample preparation for an analyte.

FIG. 3 illustrates an acoustic stimulation graph demonstrating pulse duration and period parameters of applied acoustic waves in accordance with an embodiment of the disclosed subject matter.

FIG. 4A illustrates a schematic representation of gold nanoparticles (AuNPs) functionalized with recognition molecules that aggregate in the presence of a target analyte in accordance with an embodiment of the disclosed subject matter. FIG. 4B illustrates an application of surface standing acoustic waves (SSAW) enhancing the kinetics of analyte-induced aggregation.

FIG. 5 illustrates a process of detection applying acoustic stimulation to the nanoparticles in accordance with an embodiment of the disclosed subject matter.

FIG. 6A shows representative detection results obtained by dynamic light scattering (DLS). FIG. 6B illustrates particle size distributions versus peak positions in the absence and presence of the analyte, with acoustic stimulation enabling aggregation and detection at lower analyte concentrations.

FIGS. 7A-7B present experimental data showing detection sensitivity with and without acoustic stimulation. FIG. 7A shows an acoustic period duration verse different cluster size, demonstrating detection efficiency. FIG. 7B shows an aggregation threshold shift for nano-particle clusters by several orders of magnitude when acoustic excitation is applied, demonstrating improved detection capability at low analyte concentrations.

It is to be understood that both the foregoing general description and the following detailed description are exemplary and are intended to provide further explanation of the disclosed subject matter.

### DETAILED DESCRIPTION OF THE INVENTION

The disclosed subject matter provides techniques for detecting small amounts of biological molecules using sound waves to enhance how nanoparticles aggregate together in the presence of a target. In these techniques, nanoparticles are modified with special recognition molecules, such as antibodies or DNA, that bind to the target analyte. When a sample containing the analyte is introduced, the binding causes the nanoparticles to aggregate, and the aggregation is accelerated by applying a patterned ultrasound signal, e.g., a surface standing acoustic wave (SSAW). The resulting aggregates can then be detected using simple optical techniques, such as light scattering or spectroscopy, to confirm the presence of the analyte at much lower concentrations than conventional methods allow. The disclosed subject matter also provides an apparatus including the capillary reaction chamber, a piezoelectric substrate with electrodes to generate the SSAW, and a detector for detecting the aggregation signal.

Certain biosensing platforms, including those based on nanoparticle aggregation, can be limited by the rate at which analyte molecules encounter recognition motifs on the sensor. At low analyte concentrations, this kinetic barrier prevents efficient cluster formation, resulting in weak or undetectable signals. The disclosed subject matter overcomes this limitation by introducing an acoustic field that promotes non-equilibrium interactions, thereby increasing the probability of analyte-mediated aggregation events and enabling detection at concentrations several orders of magnitude lower than certain approaches.

Reference will now be made in detail to the various exemplary embodiments of the disclosed subject matter, which are illustrated in the accompanying drawings. The structure and corresponding method of operation of the disclosed subject matter will be described in conjunction with the detailed description of the system. The accompanying drawings, where like reference numerals refer to identical or functionally similar elements throughout the separate views, serve to further illustrate various embodiments and to explain various principles and advantages all in accordance with the disclosed subject matter.

### Definitions

As used herein, the term “analyte” or “target analyte” refers to any molecule, compound, or entity whose presence or concentration is to be detected. Analytes can include, but are not limited to, DNA linkers, proteins, nucleic acids, carbohydrates, lipids, metabolites, toxins, pathogens, cells, or environmental contaminants.

As used herein, the term “acoustic wave” refers to a mechanical wave propagating through a medium (solid, liquid, or gas) that generates periodic compression and rarefaction. In the subject matter, acoustic waves can include surface acoustic waves (SAWs), standing surface acoustic waves (SSAWs), bulk acoustic waves, or other waveforms capable of producing localized pressure gradients in solution.

As used herein, the term “standing surface acoustic wave” (SSAW) refers to an acoustic wave generated when two counter-propagating surface acoustic waves of the same frequency interfere, forming stationary pressure nodes and antinodes on a substrate surface.

As used herein, the term “acoustic constant” refers to the combined effect of density and compressibility of a particle or medium that determines its acoustic contrast factor relative to the surrounding environment. Differences in acoustic constant enable particles to be manipulated or concentrated by acoustic waves.

As used herein, the term “non-equilibrium bioreaction” means a reaction or molecular interaction driven or accelerated under conditions where equilibrium thermodynamics do not dominate, such as when acoustic forces alter diffusion, collision frequency, or local concentrations of reactants.

As used herein, the term “molecular recognition” refers to the specific binding interaction between a biomolecule (e.g., antibody, aptamer, enzyme, or nucleic acid) and a target analyte (e.g., protein, nucleic acid, cell, pathogen, or another biomarker).

As used herein, the term “target analyte” means any molecule, compound, or entity to be detected, including but not limited to nucleic acids, proteins, lipids, metabolites, pathogens, toxins, or environmental contaminants.

As used herein, the term “functionalization” refers to the process of attaching a recognition molecule (e.g., antibody, aptamer, or nucleic acid) or chemical moiety to the surface of a nanoparticle or other substrate to impart specific binding properties.

As used herein, the term “limit of detection (LOD)” or “detection threshold” refers to the lowest concentration of an analyte that can be reliably distinguished from background noise under specified experimental conditions.

As used herein, the term “plasmonic coupling” means the interaction of localized surface plasmon resonances between adjacent metallic nanoparticles that alters their optical properties, such as scattering intensity or wavelength shift.

As used herein, the term “aggregation” refers to the clustering or association of two or more particles, such as nanoparticles, into larger assemblies. Aggregation can occur directly or indirectly through interactions mediated by a target analyte that binds to recognition molecules present on the particles. In certain embodiments, aggregation induced by the analyte is distinguished from nonspecific aggregation.

As used herein, the term “pulse duration” refers to the length of time during which an acoustic wave is actively applied in a single excitation event. For example, a pulse duration of 50 milliseconds (ms) indicates that the ultrasound signal is continuously transmitted for 50 ms before being turned off. The term “acoustic period” refers to the total time of one pulse cycle, including both the pulse duration (on-time) and the subsequent rest interval (off-time).

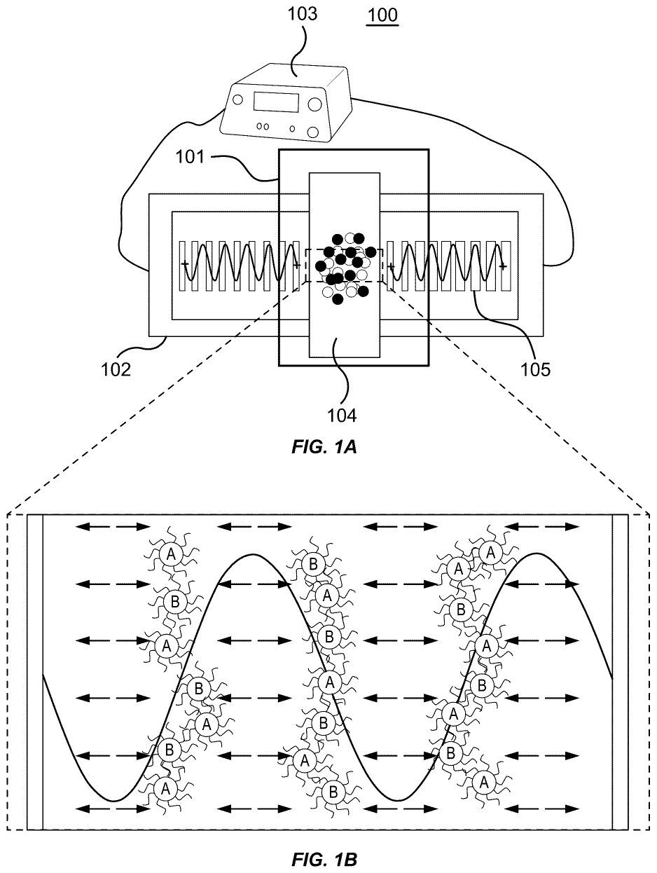

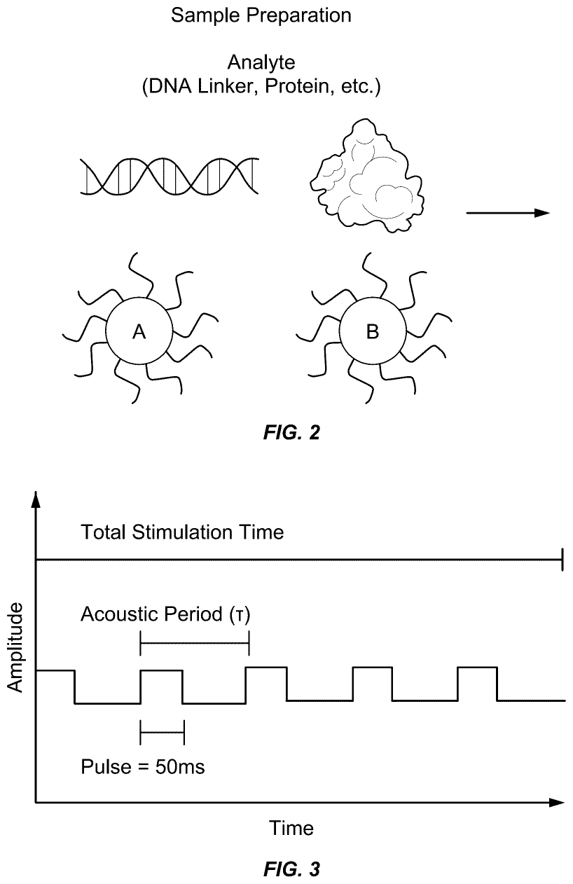

In certain exemplary embodiments, and with reference to FIGS. 1A-1B, the disclosed subject matter provides an apparatus 100 for detecting a target analyte using acoustic stimulation. The apparatus 100 includes a reaction chamber 101, a piezoelectric substrate 102, an electrical signal generator 103, and a capillary 104, a plurality of interdigital transducers (ITDs) 105, and a detector (not shown). The reaction chamber 101 comprises the capillary 104 therein, configured to receive a sample containing a target analyte such as DNA linker, protein, etc., and nanoparticles functionalized with recognition molecules, as shown in FIG. 2. The IDTs 105 can be approximately 1×0.5 cm in size with a spacing of about 0.5 cm between adjacent transducers, thereby enabling efficient coupling and propagation of acoustic waves across the substrate. In certain embodiments, the capillary 104 is designed with dimensions of approximately 1 mm×5 cm×50 μm.

In certain embodiments, the reaction chamber can be a microfluidic chamber or a small cuvette, each adapted to contain microliter- to milliliter-scale volumes of liquid samples. The piezoelectric substrate 102, for example lithium niobate (LiNbO₃), is operatively coupled to the reaction chamber 101. The electrical signal generator 103 is configured to apply an electrical signal to the piezoelectric substrate 102 to generate a surface standing acoustic wave (SSAW) within the reaction chamber 101. The capillary 104 containing the target analyte functionalized with nanoparticles is positioned on the piezoelectric substrate 102. The SSAW are generated on a piezoelectric substrate 102 by applying sinusoidal electrical signals to interdigitated electrodes equipped with IDTs 105, such that the IDTs 105 output the SSAW to the capillary 104.

The detector (not shown) is configured to operatively couple with the apparatus 300 to measure aggregation of the nanoparticles mediated by the target analyte. In certain embodiments, the capillary 104 can include glass capillary, such as a borosilicate glass tube, a fused silica capillary, or a molded polymer microchannel, each of which provides optical transparency for detection and compatibility with fluidic handling. In certain embodiments, the signal generator 103 can comprise a function generator, waveform synthesizer, or radiofrequency (RF) amplifier capable of producing sinusoidal outputs in the range of about 1 MHz to about 50 MHz. In certain embodiments, the IDT 105 can include a single-electrode IDT, double-electrode IDT, or split-finger IDT, depending on the desired bandwidth and suppression of unwanted modes. For higher efficiency, a double-electrode IDT design can be used to reduce electrical impedance.

FIG. 1B illustrates motions and aggregation propelled by acoustic waves when the waves are applied to generate the SSAW within the capillary 104. The counter-propagating waves interfere to form a standing wave pattern that establishes an acoustic field with alternating pressure nodes and antinodes. Functionalized nanoparticles suspended in the fluid are driven toward specific regions by acoustic radiation forces, which depend on their acoustic contrast factor (determined by the difference in density and/or compressibility between the particle and the surrounding medium). This mechanism creates localized regions of increased nanoparticle concentration, thereby enhancing collision frequency and accelerating receptor-analyte interactions. As a result, SSAW stimulation promotes aggregation at analyte concentrations below the conventional detection threshold.

In certain embodiments, the SSAW is generated by two counter-propagating surface acoustic waves of the same frequency and amplitude, matched in phase to ensure stable interference. The SSAW consists of alternating regions of compression and rarefaction propagating along the surface of the elastic substrate, which can be harnessed for directed assembly of particles within the reaction chamber 101. By exploiting the piezoelectric effect of the lithium niobate (LiNbO₃) crystal, the applied electrical signals are converted into acoustic waves. Experimental results confirm that SSAW application significantly enhances analyte-induced aggregation events, thereby improving the sensitivity of biosensing assays.

FIG. 3 illustrates representative acoustic stimulation profiles, including pulse duration and repetition period parameters, which affect the efficiency of molecular recognition enhancement. Acoustic stimulation can be applied either continuously or in pulsed mode. In certain embodiments, ultrasonic acoustic waves at approximately 19.42 MHz are applied in pulses of about 50 milliseconds with a repetition period of about 1 second, as shown in FIG. 3. Notably, alternative interval times can be adapted based on the properties of the nanoparticles and biomolecules.

This stimulation profile has been shown to provide optimal enhancement of analyte-induced aggregation. The pulse duration, duty cycle, and frequency can be varied or adaptively tuned depending on factors such as the target analyte, recognition motif, and fluidic configuration, as described in greater detail below.

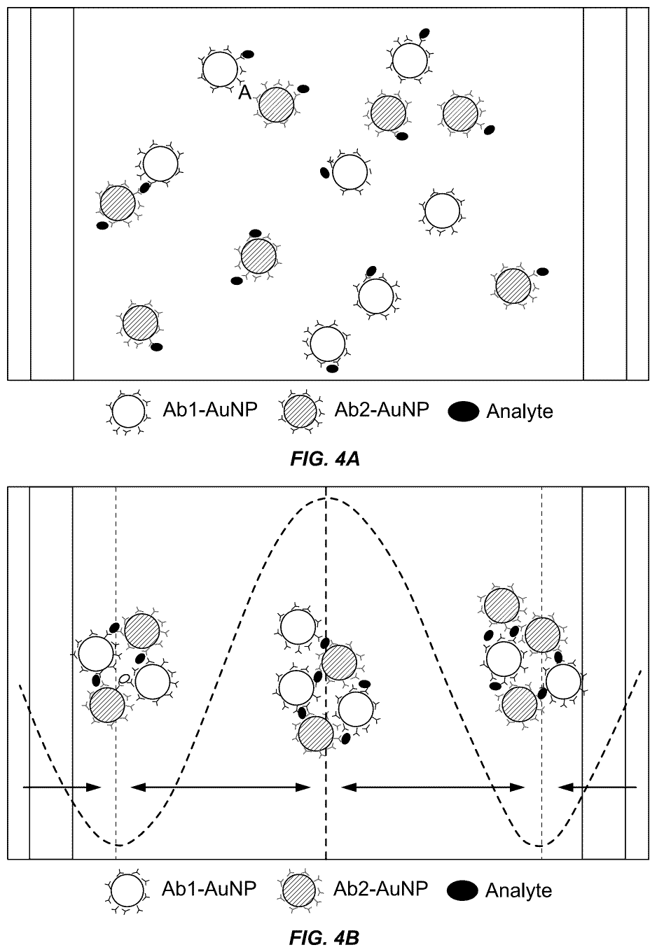

Referring to FIGS. 4A-4B, the disclosed subject matter demonstrates enhancement of reaction kinetics through application of standing surface acoustic waves (SSAW). As shown in FIGS. 4A-4B, gold nanoparticles (AuNPs) functionalized with two distinct antibodies (Ab1-AuNP and Ab2-AuNP) can aggregate in the presence of a target analyte, such as a viral nucleoprotein. In the absence of acoustic stimulation, analyte-mediated aggregation proceeds primarily through diffusion-limited encounters, resulting in slow cluster formation and higher detection thresholds. When SSAW excitation is applied, nanoparticles are actively driven into pressure nodes within the reaction chamber, resulting in localized increases in particle concentration. This directed transport accelerates binding between analyte and recognition molecules, thereby enhancing aggregation kinetics. Dynamic light scattering (DLS) measurements confirm that the hydrodynamic radius of nanoparticle clusters increases more rapidly under SSAW excitation than under quiescent conditions.

In certain embodiments, gold nanoparticles (AuNPs) with diameters of 10-200 nm are functionalized with biological recognition molecules, such as antibodies, antibody fragments, aptamers, or nucleic acids. Functionalization can be performed using covalent coupling, adsorption, or other conjugation chemistries to immobilize the recognition motif on the nanoparticle surface. As shown in FIGS. 4A-4B, analyte-induced aggregation is achieved by employing two populations of functionalized nanoparticles. For example, in certain embodiments, a first population of AuNPs can be conjugated to a first antibody (Ab1) recognizing a first epitope of the analyte, while a second population of AuNPs can be conjugated to a second antibody (Ab2) recognizing a non-overlapping epitope. In the presence of the target analyte, crosslinking occurs between Ab1-AuNPs and Ab2-AuNPs, producing clusters of nanoparticles. Such a process is facilitated and accelerated by the acoustic stimulation, leading to aggregation at analyte concentrations well below the threshold of conventional systems. Thus, the disclosed subject matter has demonstrated an enhancement on the kinetics of analyte-induced aggregation.

In certain embodiments, AuNPs can be functionalized with complementary nucleic acid sequences. For example, a first nanoparticle population carries a first DNA sequence, and a second population carries a second DNA sequence. Aggregation is induced by a linker oligonucleotide complementary to both sequences.

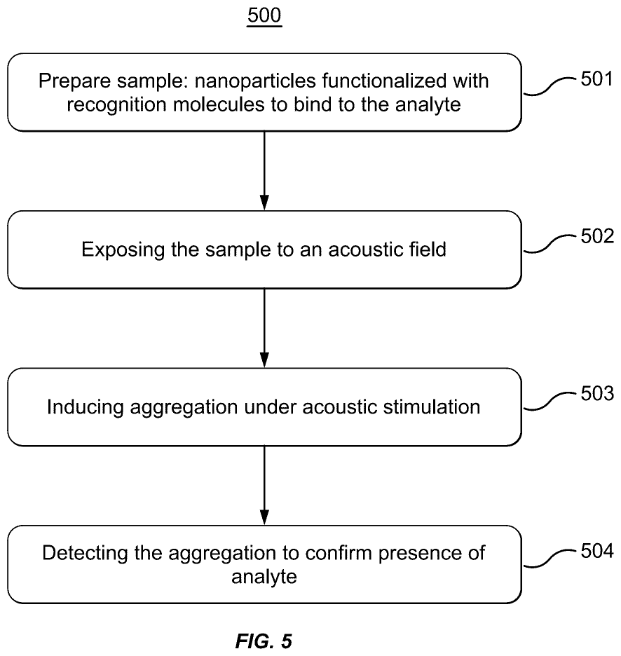

In certain embodiments, the disclosed subject matter provides a method of detecting a target analyte in a sample by employing the detection apparatus described above. An example process 500 is illustrated in FIG. 5, although the disclosed subject matter is not limited to the specific depiction therein. First, a sample is prepared at 501, where nanoparticles are provided to be functionalized with one or more recognition molecules configured to bind specifically to the target analyte. The nanoparticles can include, for example, gold nanoparticles, silver nanoparticles, silica nanoparticles, magnetic nanoparticles, or polymeric nanoparticles. Functionalization can be achieved through covalent conjugation, adsorption, or linker chemistry, and the recognition molecules can include antibodies, antibody fragments, aptamers, nucleic acids, enzymes, or combinations thereof. By tailoring the recognition molecules to the analyte of interest, the nanoparticles act as selective probes for analyte-induced aggregation. The recognition molecules can include antibodies, antibody fragments, aptamers, nucleic acids, enzymes, or combinations thereof. The target analyte can include a DNA linker, protein, nucleic acid, pathogen, toxin, metabolite, or biomarker associated with a disease state.

Following the functionalization, the sample containing the target analyte and the functionalized nanoparticles are exposed to an acoustic field at 502, where a surface standing acoustic wave (SSAW) is generated, thereby resulting in an acoustic excitation. The acoustic field can be generated by applying an electrical signal to a piezoelectric substrate, such as lithium niobate (LiNbO₃). The electrical signal induces counter-propagating acoustic waves that interfere to form a SSAW pattern with alternating pressure nodes and antinodes. Nanoparticles are driven into these nodal regions, increasing their local concentration and promoting analyte-mediated interactions.

The acoustic excitation can be applied continuously or in pulsed form. In certain embodiments, ultrasound pulses are applied with durations between about 10 milliseconds and about 100 milliseconds and with repetition periods between about 0.1 seconds and about 5 seconds. In the exemplary experiments, a pulse duration of about 50 milliseconds with a period of about 1 second provided optimal enhancement of nanoparticle aggregation. Frequencies suitable for SSAW excitation can range from about 1 MHz to about 50 MHz, depending on the substrate, fluidic geometry, and nanoparticle size.

Following the exposure to the SSAW, the aggregation of nanoparticles mediated by the target analyte is induced at 503. For example, when two populations of antibody-functionalized nanoparticles recognize distinct epitopes of the analyte, the analyte acts as a bridge, causing crosslinking and aggregation. In another embodiment, nanoparticles functionalized with complementary DNA sequences aggregate in the presence of a linker oligonucleotide serving as the analyte. The application of SSAW accelerates these aggregation events by overcoming diffusion-limited kinetics, allowing detection at analyte concentrations far below conventional thresholds.

The following detection 504 of nanoparticles aggregation is performed using one or more modalities. In certain embodiments, optical spectrophotometry is used to detect plasmonic shifts in the absorbance spectrum associated with aggregation of metallic nanoparticles. In another embodiment, dynamic light scattering (DLS) is used to measure increases in hydrodynamic radius. In further embodiments, dark-field microscopy can visualize colorimetric changes associated with nanoparticle clustering, while electron microscopy can provide structural confirmation of aggregate formation. Other detection modalities such as scattering intensity mapping, fluorescence assays, or flow cytometry can also be employed, depending on the analyte and assay format.

In certain embodiments, a parallel model method was developed using DNA-mediated nanoparticle aggregation. In this method, two populations of gold nanoparticles (AuNP-A and AuNP-B) were functionalized, e.g., with distinct single-stranded DNA sticky ends. Aggregation was triggered only in the presence of a third linker strand serving as the analyte. Initial results demonstrated that application of SSAW enhanced DNA-mediated aggregation kinetics and detection sensitivity by at least an order of magnitude relative to unstimulated samples. These results below indicate that acoustic enhancement is a general physical phenomenon applicable to a broad range of receptor-analyte pairs, including antibody-antigen and nucleic acid hybridization systems.

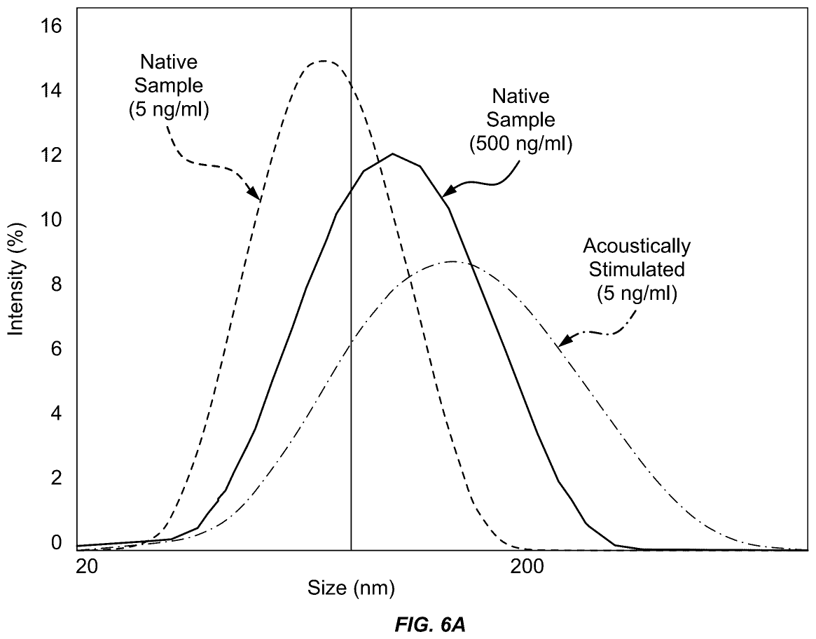

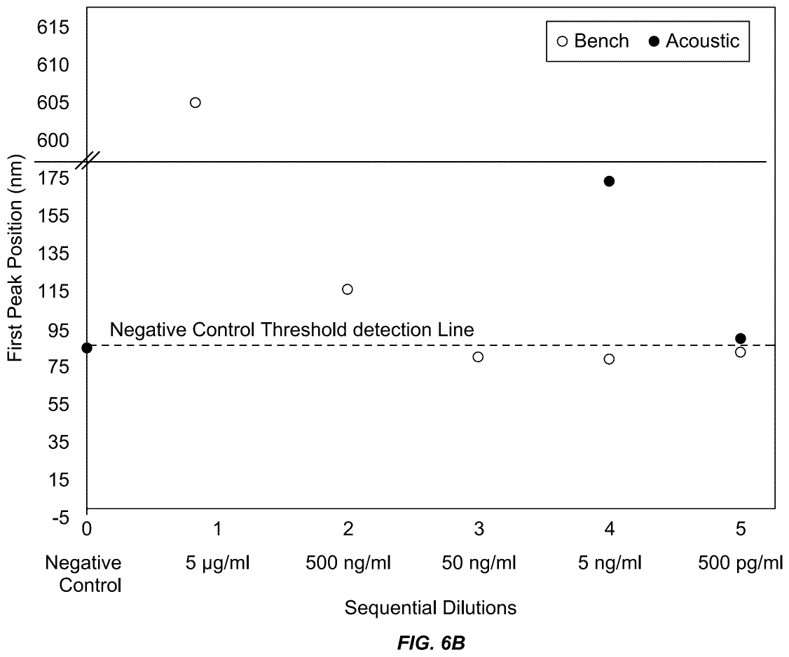

Referring to FIGS. 6A-6B, the disclosed subject matter provides acoustically enhanced detection of analytes at concentrations otherwise below the detection threshold of conventional biosensing methods. As illustrated in FIG. 6A, at concentrations lower than approximately 50 ng/ml (10.5 nM) of nucleoprotein analyte, nanoparticles functionalized with antibodies remain dispersed in solution, showing no measurable aggregation. However, when treated with SSAW, the same nanoparticle suspension exhibited aggregation in the presence of analyte at 5 ng/ml, a concentration that was undetectable without acoustic stimulation. Dynamic light scattering (DLS) measurements showed that acoustically stimulated samples produced hydrodynamic radii consistent with cluster formation, whereas native samples (unstimulated) at equivalent concentrations did not. These results demonstrate that application of acoustic energy enhances biosensor sensitivity by at least two orders of magnitude.

FIG. 6B further illustrates the detection threshold enhancement by comparing peak position shifts in DLS intensity distributions. In control samples lacking analyte, measured peak positions remained in the baseline region corresponding to individual nanoparticles. In contrast, acoustically stimulated samples containing analyte exhibited a clear transition into the aggregation region, with peak positions significantly above the negative control threshold. These results confirm that acoustic excitation facilitates earlier onset of aggregation and reduces the minimum analyte concentration required for detection by at least 100-fold, thereby improving both sensitivity and dynamic range of the assay.

These above embodiments have demonstrated that the disclosed methods and systems can be adapted to biosensing of diverse targets, including proteins, nucleic acids, pathogens, metabolites, and disease-associated biomarkers. By coupling SSAW excitation with nanoparticle-based recognition elements, analyte-induced aggregation can be accelerated and detected at concentrations significantly lower than conventional limits of detection, establishing a general platform for high-sensitivity biomolecular sensing.

### EXAMPLE

### DNA Linker, Protein-Mediated Aggregation

Two types of AuNPs were prepared by grafting distinct DNAs or proteins sticky-end sequences onto the nanoparticle surface. Aggregation required the presence of a complementary linker oligonucleotide serving as the analyte. Without acoustic stimulation, aggregation was not detectable below a threshold concentration of the linker. With SSAW excitation under pulsed conditions, aggregation was detected at concentrations at least an order of magnitude lower. These results establish that acoustic enhancement applies not only to protein-antibody interactions but also to nucleic acid hybridization.

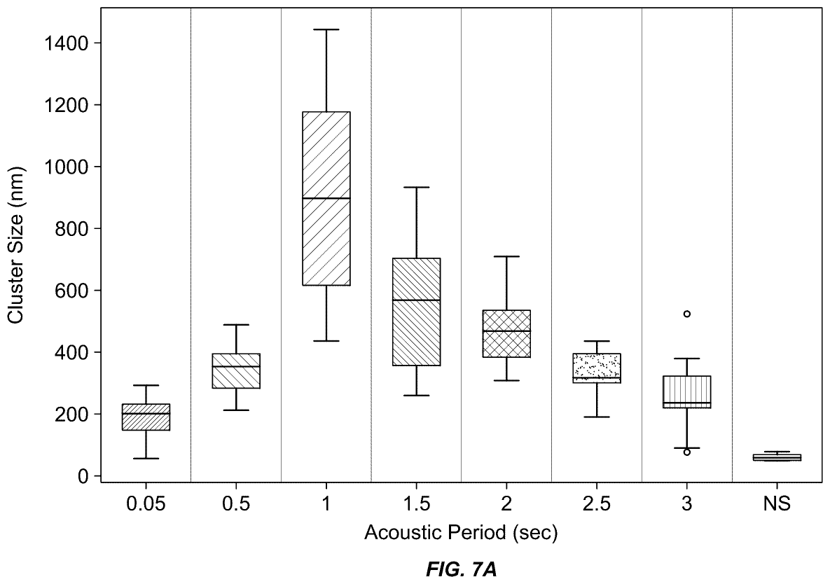

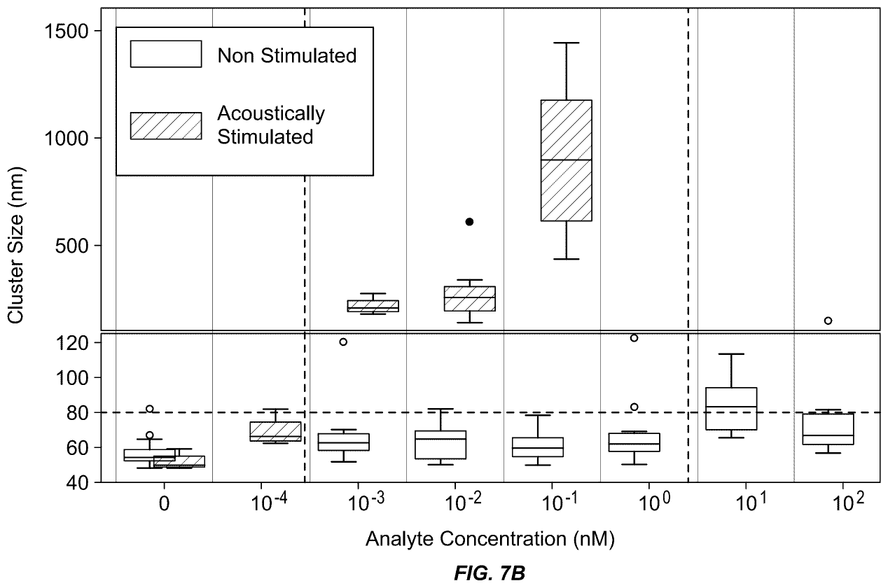

The representative results are presented illustratively in FIGS. 7A-7B, demonstrating the effect of the acoustic stimulation period on the enhancement efficiency of molecular recognition. FIG. 7A shows that varying the acoustic period produces different levels of enhancement, with a non-monotonic dependence, wherein the vertical axis corresponds to cluster size (for example, hydrodynamic diameter measured by dynamic light scattering) and the horizontal axis corresponds to the acoustic period. In exemplary experiments, SSAW are applied at a carrier frequency of approximately 19.42 MHz, with pulsed excitation profiles consisting of varying pulse lengths and repetition intervals. It was observed that a stimulation profile of 50 ms pulse duration with a 1-second period maximized enhancement of analyte-induced nanoparticle aggregation. The temporal optimization of the acoustic waveform is critical for achieving high sensitivity, indicating that the acoustic stimulation plays a decisive role in promoting efficient molecular recognition in nanoparticle-based biosensing. Notably, the cluster size of the functionalized nanoparticles can be up to around 1000 nm under stimulations of 1-second acoustic period, compared the cluster size under 600 nm for the samples stimulated with fewer or more periods. In contrast, the cluster size of the samples without the acoustic stimulation (NS) is under 200 nm.

FIG. 7B reports detection sensitivity measurements that compare nanoparticle aggregation in the absence and presence of optimized SSAW stimulation. In FIG. 7B, the vertical axis corresponds to cluster size (for example, hydrodynamic diameter measured by dynamic light scattering) and the horizontal axis corresponds to analyte concentration. A green horizontal dotted line in the figure indicates the minimum cluster-size threshold used to confirm analyte presence. A gray vertical dotted line (right) indicates the practical detection limit observed for unstimulated (bench) samples, while a pink vertical dotted line (left) indicates the lowered detection limit observed for samples subjected to SSAW. The data show that, without acoustic stimulation, aggregation was not observed below relatively high analyte concentrations, for example, in the micromolar to high-nanomolar range under the tested conditions. By contrast, with SSAW stimulation, detectable aggregation was observed at analyte concentrations several orders of magnitude lower, including concentrations in the single-digit picomolar range in certain embodiments. Overall, these results confirm that SSAW stimulation can lower the minimum detectable analyte concentration by at least two orders of magnitude (≥100×), and in certain embodiments by up to four orders of magnitude (≈10,000×), thereby enabling ultra-sensitive detection that is not achievable under diffusion-limited, unstimulated conditions.

In certain alternative embodiments, the systems and methods of the disclosed subject matter can be applied to the detection of viral proteins, including the severe acute respiratory syndrome coronavirus 2 (SARS-CoV-2) nucleoprotein. In such embodiments, the detection can be processed based on gold nanoparticles (AuNPs) functionalized with monoclonal antibodies directed against either the SARS-CoV-2 nucleoprotein or spike protein. The detection assesses whether analyte-induced aggregation of functionalized nanoparticles occurs, thereby enabling detection sensitivity down to the level of single nanoparticle clusters using inexpensive optical characterization techniques.

In certain embodiments, the specific recognition between antibody-conjugated AuNP complexes and their corresponding antigens, combined with the size-dependent optical properties of the nanoparticles (including scattering intensity, spectroscopic signature, and polarization of scattered light), is utilized for target detection. For example, two distinct monoclonal antibodies recognizing non-overlapping epitopes of the SARS-CoV-2 nucleoprotein were conjugated to separate populations of AuNPs (Ab1-AuNPs and Ab2-AuNPs, respectively). Upon exposure to the nucleoprotein analyte, crosslinking between the Ab1-AuNPs and Ab2-AuNPs is induced, resulting in aggregation. The plasmonic coupling between adjacent antibody-functionalized AuNPs produces a measurable optical signal, rendering the system highly sensitive for biosensing applications. Using this detection, limits of rapid detection based on plasmonic particle signatures were established, demonstrating visible differences in the presence and absence of the target analyte. This detection system was further utilized to investigate the effect of acoustic wave stimulation on the kinetics of analyte-induced aggregation, thereby serving as a proof-of-concept for acoustically enhanced biosensing of viral biomarkers.

The disclosed subject matter is compatible with numerous biosensing platforms, including lateral flow assays, microfluidic chips, and benchtop spectroscopic devices. Integration requires only the addition of an acoustic stimulation module and does not necessitate redesign of existing recognition chemistries or transduction schemes.

While the above-discussed embodiments relate to biosensing, the acoustic enhancement approach can also be applied to other molecular processes in which reaction kinetics limit efficiency. These include protein crystallization, supramolecular self-assembly, and enzymatic catalysis. By providing localized energy input and inducing concentration gradients, acoustic fields can accelerate molecular interactions in a broad range of systems.

The foregoing merely illustrates the principles of the disclosed subject matter. Various modifications and alterations to the described embodiments will be apparent to those skilled in the art in view of the teachings herein. Further, it should be noted that the language used herein has been selected for readability rather than to delineate or limit the disclosed subject matter. Accordingly, the disclosure herein is intended to be illustrative, but not limiting, of the scope of the disclosed subject matter. Moreover, the principles of the disclosed subject matter can be implemented in various configurations of hardware and/or software and are not intended to be limited in any way to the specific embodiments presented herein. It will thus be appreciated that those skilled in the art will be able to devise numerous systems, arrangements, and procedures which, although not explicitly shown or described herein, embody the principles of the disclosure and can be thus within the spirit and scope of the disclosure.

What is claimed is:

1. A method of detecting a target analyte in a sample, comprising:
providing nanoparticles, functionalized with one or more recognition molecules configured to bind specifically to the target analyte, to the sample;
exposing the sample with the target analyte and the functionalized nanoparticles to an acoustic field comprising a surface standing acoustic wave (SSAW);
inducing aggregation of the nanoparticles mediated by the target analyte under acoustic stimulation of the SSAW; and
detecting the aggregation of the nanoparticles as an indication of presence of the target analyte.

2. The method of claim 1, wherein the acoustic field is generated by applying an electrical signal to a piezoelectric substrate.

3. The method of claim 1, wherein the acoustic field is configured to generate acoustic excitation having a frequency between about 1 MHz and about 50 MHz.

4. The method of claim 1, wherein the acoustic stimulation is applied in pulses having a duration of about 10 ms to about 100 ms with a period of about 0.1 s to about 5 s.

5. The method of claim 1, wherein the recognition molecules comprise at least one of antibodies, antibody fragments, aptamers, nucleic acids, or enzymes.

6. The method of claim 1, wherein the target analyte comprises at least one of DNA linker, protein, nucleic acid, pathogen, toxin, metabolite, or biomarker associated with a disease state.

7. The method of claim 1, wherein the detecting comprises at least one of optical detection, dynamic light scattering, dark-field microscopy, spectrophotometry, or electron microscopy.

8. The method of claim 1, wherein the SSAW is generated by a plurality of interdigital transducers (IDTs) patterned on a piezoelectric substrate.

9. The method of claim 1, further comprising tuning pulse duration and acoustic period to adjust aggregation efficiency.

10. The method of claim 1, wherein the nanoparticles are functionalized with two or more recognition molecules binding to non-overlapping epitopes of the analyte.

11. The method of claim 1, wherein the detecting comprises monitoring a plasmonic shift in optical spectra associated with aggregation of the nanoparticles.

12. An apparatus for detecting a target analyte in a sample, comprising:
a reaction chamber having a capillary configured to receive a sample containing a target analyte and nanoparticles functionalized with recognition molecules;
a piezoelectric substrate operatively coupled to the reaction chamber;
an electrical signal generator configured to apply an electrical signal to the piezoelectric substrate to generate a surface standing acoustic wave (SSAW) within the capillary; and
a detector configured to measure an aggregation of the nanoparticles mediated by the target analyte.

13. The apparatus of claim 12, wherein the detector comprises at least one of an optical detector, spectrophotometer, dark-field microscope, or dynamic light scattering instrument.

14. The apparatus of claim 12, wherein the nanoparticles comprise gold nanoparticles conjugated to two or more antibodies recognizing specific epitopes of the target analyte.

15. The apparatus of claim 12, wherein the piezoelectric substrate comprises lithium niobate (LiNbO₃).

16. The apparatus of claim 12, wherein the capillary comprises a glass capillary mounted on the piezoelectric substrate.

17. The apparatus of claim 12, wherein the detector is configured to detect changes in hydrodynamic radius of nanoparticle clusters using dynamic light scattering.

18. The apparatus of claim 12, wherein the detector is configured to output a visual indication of aggregation at analyte concentrations at least 100-fold lower than detection in absence of acoustic stimulation.

19. The apparatus of claim 12, wherein the electrical signal generator is configured to apply electrical signals to an interdigital transducer to generate counter-propagating acoustic waves.

20. The apparatus of claim 19, wherein the electrical signals are configured to vary at least one of frequency, amplitude, or duty cycle to adaptively tune the SSAW.
